(19) United States
(12) Patent Application Publication
(10) Pub. No.: US 2011/0287415 A1
(43) Pub. Date: Nov. 24, 2011

(54) IN-SITU HYBRIDIZATION TO DETECT RNA AND DNA MARKERS

(75) Inventors: Wen-Hua Fan, San Diego, CA (US); Roger Tim, La Jolla, CA (US); Ram Bhatt, San Diego, CA (US)

(73) Assignee: Novartis AG, Basel (CH)

(21) Appl. No.: 12/671,902

(22) PCT Filed: Aug. 4, 2008
(86) PCT No.: PCT/US2008/072143

§ 371 Date: May 20, 2011

Related U.S. Application Data
(60) Provisional application No. 60/953,812, filed on Aug. 3, 2007.

(51) Int. Cl. C12Q 1/68; C07H 21/04

(52) U.S. Cl. 435/6.11; 536/24.31

(57) ABSTRACT

The present invention provides probes, kits and methods for specifically binding mRNA of target cells, e.g., fetal cells, that allows visualization of the target cells, under conditions that allows a second probe to specifically bind the target cell's chromosomal DNA.

### CROSS-REFERENCE TO RELATED APPLICATION

This application claims priority to U.S. Provisional Patent Application Ser. No. 60/953,812, filed Aug. 3, 2007, entitled “In-situ Hybridization to Detect RNA and DNA Markers”.

### BACKGROUND OF THE INVENTION

There are essentially two types of fetal cell biomarkers; antibodies for the fetal cell surface antigens, and cytoplasmic proteins and gene expression markers for genes preferably expressed in fetal cells than in maternal cells. Antibody markers can be labeled directly or indirectly and visualized using fluorescence techniques. However, the two problems with this method are that the autofluorescence of heme interferes with the fluorescence signal of fetal cells stained positive for antibody binding; and the non-specificity of the antibodies which generally cross reacts with antigens on the maternal cells resulting in tremendous background that severely interferes with the signal generated by the fetal cells. The gene expression markers have their own problem—the targeted RNA is generally unstable and thus not available for binding with the ligand.

Currently known methods for visualizing fetal cells include staining the cells. The stains used for this purpose interfere with, and thereby pose a problem, for further analysis with fluorescence in-situ hybridization (FISH).

There is a need to develop more methods, e.g., non-invasive methods for detecting fetal cells, especially genetic and/or gene markers associated with fetal cells.

### SUMMARY OF THE INVENTION

The present invention is based, in part, on the discovery that one can detect RNA markers as well as DNA markers in the same cell, especially under conditions allowing in situ detection of RNA markers as well as DNA markers. Accordingly, the present invention provides probes, kits and methods useful for detecting RNA and DNA markers as well as genetic disorders associated therewith.

In one embodiment of the invention, it provides a method for detecting mRNA and chromosomal DNA in a cell. The method comprises in situ hybridization of a first probe and a second probe to a biological sample containing one or more target cells. The hybridization takes place under conditions that allow the first probe to specifically hybridize to its mRNA target in a target cell and the second probe specifically hybridize to its chromosomal DNA target in the target cell. In general, the duplex formed between the first probe and its mRNA target has a Tm of at least about 80° C. Alternatively, the first probe has a GC content of at least about 40%.

In another embodiment of the invention, it provides a method for detecting a genetic disorder of a fetus. The method comprises in situ hybridization of a first probe and a second probe to a biological sample containing one or more fetal cells from the fetus. The hybridization is conducted under conditions that allow the first probe to specifically hybridize to its mRNA target in a fetal cell and the second probe to specifically hybridize to its chromosomal DNA target in the fetal cell. In general, the duplex formed between the first probe and its mRNA target has a Tm of at least about 80° C., or, alternatively, the first probe has a GC content of at least about 40%. The hybridization of the first probe to its mRNA target and the second probe to its chromosomal DNA target is indicative of a genetic disorder of the fetus.

In yet another embodiment of the invention, it provides a probe that specifically hybridizes to an mRNA target in a fetal cell.

In still another embodiment of the invention, it provides a kit comprising a probe of the invention.

### BRIEF DESCRIPTION OF THE FIGURES

FIG. 1 shows the in situ hybridization of the probe to mRNA in the cell and the detection of the mRNA-bound probe.

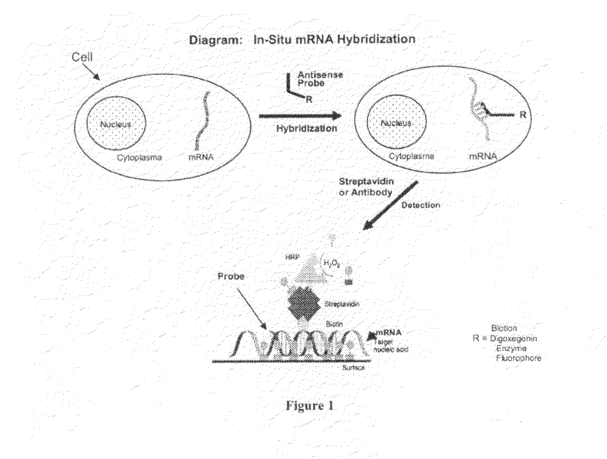

FIG. 2 shows exemplary modified nucleotides included in a probe that specifically binds to an mRNA target.

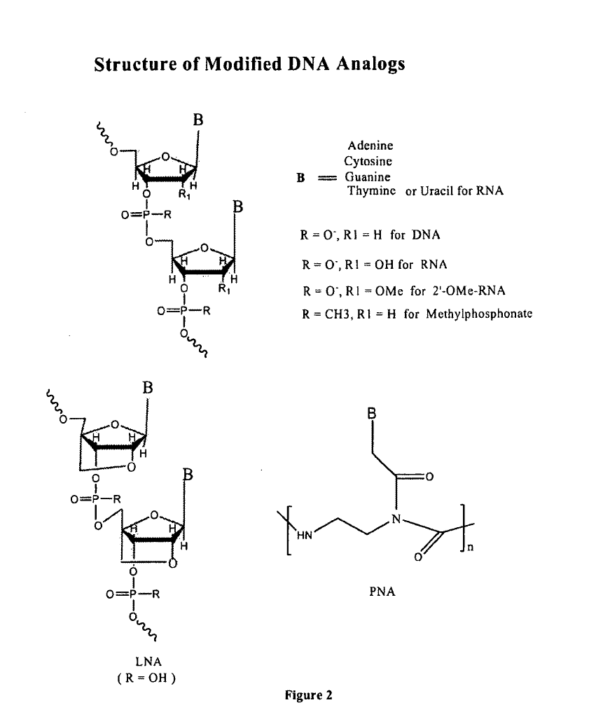

FIG. 3 is a list of exemplary probes of the invention and some their characteristics. Small letters indicate normal DNA bases whereas capital letters indicate locked nucleic acid (LNA) bases.

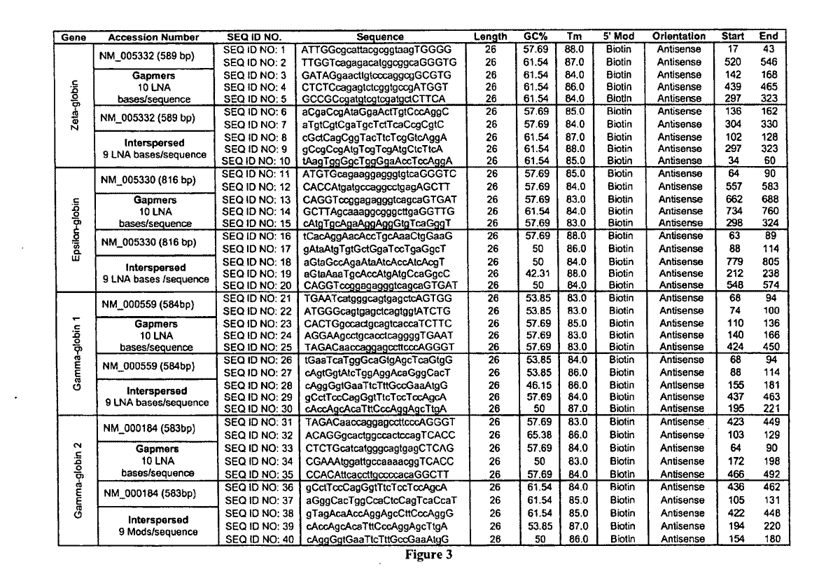

FIG. 4 is an exemplary flow chart for in situ mRNA hybridization and the subsequent detection of fetal cells.

FIGS. 5A and 5B are fluorescence pictures of fetal cells that are both mRNA and FISH positive. FIG. 5A, represents the staining of four fetal nRBCs with DIG labeled epsilon-mRNA-probe wherein the DIG is bound to anti-DIG-monoclonal antibody conjugated to Texas-Red. FIG. 5B represents the FISH signal from the same four nRBCs after staining with DAPI.

FIGS. 6A and 6B are fluorescence pictures of fetal cells that are both mRNA and FISH positive. FIG. 6A represents three epsilon-mRNA-probe stained fetal nRBCs while FIG. 6B shows the FISH XY signal from the same three cells after DAPI staining.

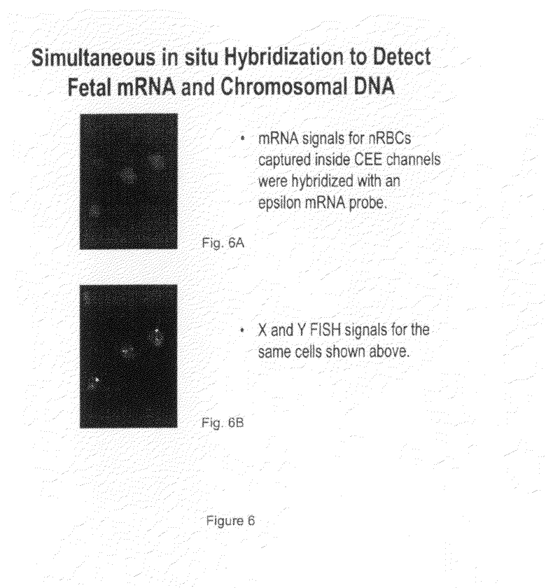

### DETAILED DESCRIPTION OF THE INVENTION

As used herein, the following terms shall have the following meanings:

The terms “polynucleotide,” “oligonucleotide,” and “nucleic acid” have the same meaning and are used interchangeably throughout. They refer to a single-stranded, directional nucleotide polymer made of more than 2 nucleotide subunits covalently joined together from a 5′-end to a 3′-end.

The abbreviations used throughout the specification to refer to polynucleotides or nucleic acids comprising specific nucleobase sequences are the conventional one-letter abbreviations. Thus, when included in a polynucleotide, the most common naturally occurring encoding nucleobases are abbreviated as follows: adenine (A), guanine (G), cytosine (C), thymine (T) and uracil (U). Unless specified otherwise, single-stranded nucleic acid sequences are represented as a series of one-letter abbreviations.

Two sequences are said to be “complementary” when the sequence of one can hybridize to the sequence of the other in an anti-parallel sense to form a “duplex” or “double strand” wherein the 3′-end of each sequence binds to the 5′-end of the other sequence and each A, T(U), G, and C of one sequence is then aligned with a T(U), A, C, and G, respectively, of the other sequence.

Conventional techniques of molecular biology and nucleic acid chemistry, which are within the skill of the art, are fully explained in the literature. See, for example, Sambrook et al., 1989, Molecular Cloning—A Laboratory Manual, Cold Spring Harbor Laboratory, Cold Spring Harbor, N.Y.; Oligonucleotide Synthesis (M. J. Gait, ed., 1984); and Nucleic Acid Hybridization (B. D. Hames and S. J. Higgins. eds., 1984).

The present invention is based, in part, on the discovery that one can detect RNA markers as well as DNA markers in the same cell, especially under conditions allowing in situ detection of RNA markers as well as DNA markers. Accordingly, the present invention provides probes, kits and methods useful for detecting RNA and DNA markers as well as genetic disorders associated therewith.

According to one aspect of the present invention, it provides methods for detecting one or more RNA markers as well as DNA markers in the same cell by hybridizing probes under a detecting condition, e.g., allowing the formation of stable RNA/probe duplex as well as DNA/probe duplex in situ.

According to the present invention, such detecting condition includes any suitable means to allow probes to hybridize, e.g., in situ to their respective RNA and DNA targets. In one embodiment, the detecting condition of the present invention includes using a probe directed to an RNA marker that is capable of forming a thermo-stable RNA/probe duplex under a denaturing condition required by a FISH assay of a DNA marker. For example, such probe can be a probe with a Tm of at least 70° C., 71° C., 72° C., 73° C., 74° C., 75° C., 76° C., 77° C., 78° C., 79° C., 80° C., 85° C., or 90° C. In another example, such probe can be a probe with a GC content of at least 20%, 25%, 30%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 70% or 75%.

In general, the probe of the present invention can be any oligonucleotide or polynucleotide with modified and/or non-modified nucleic acids. For example, the probe of the present invention can include modified and/or non-modified deoxy-nucleotides or ribo-deoxynucleotides. Exemplary modified nucleic acids include, without any limitation, locked nucleic acid (LNA), peptide nucleic acid (PNA), nucleic acid containing 2′-OMe nucleotides, nucleic acid containing methylphosphonates, nucleic acid containing phosphorothioates, and nucleic acid containing morpholino.

Usually a modified nucleic acid provides a more stable interaction with its complementary nucleic acid than that of its non-modified version. Thus the extent of modified nucleic acids contained in the probe of the present invention depends on, among other factors, the stability requirement, e.g., thermo-stability requirement of the probe. In one embodiment, the probe of the present invention is a chimeric probe, comprising deoxy-nucleotides or ribo-deoxynucleotides as well as modified deoxy-nucleotides or ribo-deoxynucleotides. In another embodiment, the modified nucleic acids are located at one or both ends of the probe of the present invention. In yet another embodiment, the modified nucleic acids are located throughout the probe of the present invention. In still another embodiment, the probe of the present invention contains about 10%, 20%, 25%, 30%, 35%, 40%, 45%, 50%, 55%, 60%, 65%, 70%, or 80% of modified nucleic acids. In yet another embodiment, the probe of the present invention contains about 10%, 15%, 20%, 25%, 30%, 35% or 40% of modified nucleic acids.

According to the present invention, the length of the probe can vary based on its target and other factors affecting the detecting condition of the present invention. In one embodiment, the probe of the present invention is directed to an RNA marker and its length is from about 10 nucleotides to about 50, 100, 200, 300, or 400 nucleotides. In another embodiment, the probe of the present invention is directed to an RNA marker and its length is from about 20 nucleotides to about 25, 26, or 27 nucleotides. In yet another embodiment, the probe of the present invention is directed to an RNA marker and its length is from about 30 nucleotides to about 40, 45, or 46 nucleotides. In general, the probe of the present invention directed to a DNA marker, for example, a nuclear DNA marker, can be any probe suitable for a FISH assay, e.g., any mixture of probes used for FISH detection of a genetic locus.

Usually the probe of the present invention is labeled directly or indirectly with a detectable entity. For example, the probe of the present invention can be labeled terminally, e.g., at either or both ends, or internally, e.g., throughout the probe. In one embodiment, the probe of the present invention can be labeled with a directly or indirectly detectable entity which emits light through any mechanism, for example, through an excitation-emission mechanism or through a chemiluminescence mechanism. In another embodiment, the probe of the present invention is labeled with a direct label selected from the list including, but not limited to, fluorescein; Cy3; Cy5; Cy7; Texas Red; StarBright Green; StarBright Orange; rhodamine green; Oregon Green; bodipy fluorophores; Alexa Fluor dyes with emission between 350 and 750 nm; and combinations thereof.

In yet another embodiment, the probe of the present invention is labeled with an indirect label selected from the list including, but not limited to, digoxigenin; biotin; Lucifer yellow; dinitrophenyl (DNP); enzyme molecules such as horseradish peroxidase (HRP), alkaline phosphotase (AP), luciferase or galatosidase; dansyl; and combinations thereof. Such probe can be visualized either with streptavidin (for a biotinylated probe) or an antibody (e.g., an anti-DiG-antibody for a digoxigenin probe) conjugated with fluorophores. Similarly, if the probe is labeled with an enzyme molecule, such probe can be visualized by using an appropriate enzyme fluorescence substrate.

According to the present invention, the probe of the present invention can be either a perfect match to its target or contain one or more mismatches to its target, e.g., 1%, 2%, 3%, 4% or 5% mismatch. In general, the probe of the present invention directed to a DNA marker is a mixture of probes usually used for FISH assay for a genetic marker or locus. With respect to the probe of the present invention directed to an RNA marker, it can be targeted to any RNA marker, e.g., an RNA marker indicating the origin, nature, identity, or stage of a target cell.

In one embodiment, the probe of the present invention includes probes targeted to one or more RNA markers in a fetal cell as well as DNA markers, e.g., genetic markers or loci. In another embodiment, the probe of the present invention includes a probe directed to an RNA marker in a fetal cell as well as a probe directed to a DNA marker associated with a genetic disorder. In yet another embodiment, the probe of the present invention includes a probe directed to an RNA marker associated with certain developmental stage of a fetus, e.g., epsilon- or zeta-hemoglobin mRNA expressed usually in 7-11 weeks old fetus or gamma-hemoglobin mRNA expressed usually in fetuses older than 11 weeks. In still another embodiment, the probe of the present invention includes a probe directed to an intracytoplasmic mRNA marker that allows the cytology and morphology based identification of cells. In still another embodiment, the probe of the present invention includes a probe directed to an mRNA marker expressed during a certain gestational period, i.e., during the first, second or third trimester. In still another embodiment, the probe of the present invention includes a probe directed to an mRNA marker expressed in a fetus during the first trimester of pregnancy. In yet another embodiment, the probe of the present invention includes a probe directed to mRNA marker expressed in a fetus between about week 5, 6 or 7 to about week 11, 12 or 13 of pregnancy.

Exemplary RNA markers for a fetal cell include, but are not limited to, epsilon-hemoglobin mRNA; zeta-hemoglobin mRNA; gamma-hemoglobin mRNA; Y chromosome-specific zinc finger protein mRNA (ZFY-mRNA); fetal alpha-fetoprotein (AFP) mRNA; gamma-glutamyl-transpeptidase (GGT) mRNA; human placental lactogen 1, 2, 3, and 4 (hPL, aka PLAC-1-4) mRNA; pregnancy associated plasma protein-A (PAPP-A) mRNA; corticotropin hormone-releasing factor (CRH) mRNA; tissue factor pathway inhibitor 2 (TFPI-2) mRNA; KISS-1 metastatic-suppressor mRNA; and PLAC-1 mRNA; pregnancy specific beta-1-glycoprotein-2, 3, 4, 5, 6, 7 and 9 mRNAs; chorionic somatomammotropin hormone 2 and variants (v1, v2, v3, v4, v5) mRNAs; growth hormone variant mRNA; alfa-disintegrin and metalloproteinase domain variants mRNAs; dipeptidylpeptidase 7 mRNA; fibulin mRNA; s100 calcium binding protein mRNA; prostate differentiation factor mRNA; and cytochrome P450, subfamily xix mRNA. Exemplary probes of the present invention directed to an RNA marker of a fetal cell includes, without any limitation, probes comprising a nucleic acid sequence selected from SEQ ID NOs. 1-40.

According to the present invention, the probe of the present invention directed to an RNA marker can be hybridized to its target either concurrently with or sequentially to the probe directed to a DNA marker, e.g., probes to an RNA marker hybridize to their target(s) prior to, during and/or after probes to a DNA marker hybridize to their target(s). In general, probes of the present invention directed to RNA markers and DNA markers can be handled under the same condition, e.g., they can be hybridized to their targets under the same denaturing, hybridization, and washing condition. In one embodiment, the hybridization takes place under conditions that allow one or more probes to RNA markers to hybridize and remain hybridized to their RNA targets while one or more probes hybridize to their DNA targets.

According to another embodiment of the present invention, the detecting condition of the present invention includes one or a combination of conditions associated with hybridization condition, wash condition, wash buffer, fixation of cells, denaturing condition, etc. In one embodiment, the detecting condition of the present invention includes a minimum hybridization time of at least 1, 2, 3, 4, 5, or 6 hours at a temperature from about 30° C., 35° C., 37° C., to about 45° C., 50° C., 55° C., 60° C., or 65° C. In another embodiment, the detecting condition of the present invention includes a post-hybridization wash process with less stringency, e.g., low temperature wash (e.g., varying from room temperature to about 80° C.), using a washing solution with 1× or 2×SSC, and optionally from about 25% to about 75% formamide with or without commonly used detergents.

In yet another embodiment, the detecting condition of the present invention includes pre-hybridization fixation of cells, e.g., for at least 15, 20, 25, 30, 35, or 40 minutes. For example, cells can be treated with about 1% to about 4% paraformaldehyde in PBS at about 25° C. to about 37° C. for about 30 to about 60 minutes. In another example, cells can be treated with about 4% paraformaldehyde in PBS at room temperature for at least 30 minutes. In yet another example, after initial treatment cells can be optionally treated with about 0.01% to about 1% Triton X-100 at about 25° C. to about 37° C. for about 2 to about 30 minutes to permeabilize them. In still another example, the cells can be permeabilized by treating with 0.01% to about 5% proteinase K under various conditions, e.g., pH 2 to 7 for a period of time.

In still yet another embodiment, the detecting condition of the present invention includes using one or more blocking agents to prevent or reduce non-specific hybridization. For example, this step can generally be carried out prior to detecting the hybridization of the probe to its target. Any compound capable of preventing or minimizing the detection of non-specific background hybridization is suitable for this purpose. An exemplary list of agents include, but are not limited to, BSA, casein, Perice's SuperBlock and Fc blocker.

According to the present invention, the method of the present invention for detecting RNA as well as DNA markers can be conducted in any target cell from a biological sample. In one embodiment, the biological sample is a cell or tissue sample, e.g., suspected of or potentially containing one or more target cells. In another embodiment, the biological sample is a bodily fluid, including, but not limited to, blood, plasma, serum, saliva, urine, spinal fluid, bone marrow, or a cervical mucous sample. In another embodiment, the biological sample is any sample potentially or suspected of containing neoplastic cells, for example, one or more tumor or cancer cells.

In yet another embodiment, the target cell is a cell from a fetus, e.g., nRBC, lymphocyte, trophoblast, etc., and the biological sample is a sample from the maternal host of the fetus containing one or more fetal cells. Any maternal host sample known or later discovered to contain fetal cells can be used for the present invention. For example, the maternal host sample that can be used as a source for fetal cells includes, without any limitation, blood, plasma, serum, saliva, urine, or a cervical mucous sample.

In yet another embodiment, the target cell is a fetal cell from a fetus and the biological sample is a sample from a maternal host of the fetus that has been enriched for fetal cells. Such a fetal cell enrichment sample can be obtained from any maternal biological sample known, or later discovered, to contain fetal cells, including, but not limited to, blood, plasma, serum, saliva, urine, and cervical mucous. In one embodiment, the fetal cell enrichment sample is obtained from the blood sample of the maternal host of the fetus.

The biological sample from the maternal host can be enriched for fetal cells by any means now known, or later discovered. In one embodiment, the maternal biological sample is contacted with a ligand that binds maternal cells in preference to fetal cells. As a consequence, the ligand removes maternal cells from the biological sample, thereby enriching the biological sample for fetal cells. In another embodiment, the maternal biological sample is contacted with a ligand that binds fetal cells in preference to maternal cells. The fetal cell-enriched sample can then be obtained by washing the fetal cells from the ligand to which they are bound under conditions and using buffers suitable for separating the ligand and fetal cell. Such conditions and buffers are known to one of skill in the art.

According to another aspect of the present invention, the method of the present invention for detecting RNA markers as well as DNA markers can be used to detect any condition associated with a genetic marker, especially for detecting any genetic condition of a cell that requires the identification or confirmation of the cell type at the same time. In one embodiment, the method of the present invention can be used to detect a neoplastic condition associated with a genetic marker. For example, the probe of the present invention directed to an RNA marker can be a probe for an RNA marker of the cell type of a target cell while the probe directed to a DNA marker can be a probe for a genetic condition susceptible or indicative of a neoplastic condition of the same target cell.

In another embodiment, the method of the present invention can be used to detect a fetus condition associated with a genetic marker. For example, the probe of the present invention directed to an RNA marker can be a probe for the identification of a fetal cell while the probe directed to a DNA marker can be a probe for a genetic condition of a fetus, e.g., chromosomal composition or genetic composition of a fetus. In yet another embodiment, the method of the present invention can be used to detect genetic disorders of a fetus. For example, probes of the present invention directed to one or more RNA markers of a fetal cell and DNA markers associated with a genetic disorder can be used according to the method of the present invention to detect one or more genetic disorders of a fetus, e.g., chromosome deletion, addition, translocation, and gene mutation, deletion, duplication, etc.

Exemplary genetic disorders include, but are not limited to, Cystic Fibrosis, Sickle-Cell Anemia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, and RhD Syndrome.

According to the present invention, the genetic disorder of the fetus can be detected at any time during the gestational period, i.e., during the first, second or third trimester. In one embodiment, the genetic disorder is detected during the first trimester of pregnancy. In another embodiment, the genetic disorder is detected between about week 5, 6 or 7 to about week 11, 12 or 13 of pregnancy. In yet another embodiment, the genetic disorder is detected when the fetus is at least five, or six, or seven weeks old.

In general, hybridization of a probe to its RNA target and a probe to its DNA target in the method of the present invention is indicative of a condition associated with the either or both targets. In one embodiment, the presence of either the RNA target or the DNA target or both as indicated by the hybridization of the probes to their respective targets is indicative of a condition associated with either or both targets. In another embodiment, the intensity or quantity with respect to the presence of the RNA target or the DNA target or both as indicated by the hybridization signal or pattern, e.g., number or intensity of hybridization signals is indicative of a condition associated with either or both targets. For example, a probe could contain a sequence which recognizes, e.g., hybridizes to a sequence in an RNA target or DNA target that is associated with a disorder or condition. Alternatively, a probe could contain a sequence which recognizes, e.g., hybridizes to a sequence in an RNA target or DNA target and where the presence of more or less than normal number of copies of such sequence is associated with a disorder or condition. For example, normal fetal cells should contain two copies of chromosome 21. Any hybridization using the method of the present invention that indicates three copies of chromosome 21 is indicative of a genetic disorder associated with having extra copies of chromosome 21, e.g., Down syndrome.

According to another aspect of the present invention, it provides kits useful for the methods of the present invention. In one embodiment, the kit of the present invention comprises one or more probes of the present invention. For example, the kit of the present invention can include one or more probes directed to an RNA marker for a particular cell type, and optionally include one or more probes directed to a DNA marker, e.g., including DNA FISH probes known and/or available such as FISH probes for genes associated with a genetic condition or FISH probes for chromosomes 13, 18, 21, X, and Y.

In another embodiment, the kit of the present invention comprises one or more probes directed to one or more RNA markers and an instruction for using such probes in connection with one or more probes directed to one or more DNA markers, e.g., according to the method of the present invention.

In yet another embodiment, the kit of the present invention comprises one or more probes of the present invention and one or more buffers useful for the method of the present invention. For example, the kit of the present invention can include one or more probes of the present invention and a buffer useful for the process of fixation, denaturing, hybridization, and/or washing, e.g., according to the method of the present invention. The kit of the present invention can additionally include one or more blocking agents or any other agents useful for the method of the present invention.

In still another embodiment, the kit of the present invention comprises one or more probes of the present invention and a sample enriched for a particular cell type of interest, e.g. fetal cells enriched from maternal biological samples or neoplastic cells enriched from a biological sample of a host subject.

### EXAMPLES

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### Example 1

### In Situ Hybridization of Fetal mRNA and the Detection of Fetal Cells

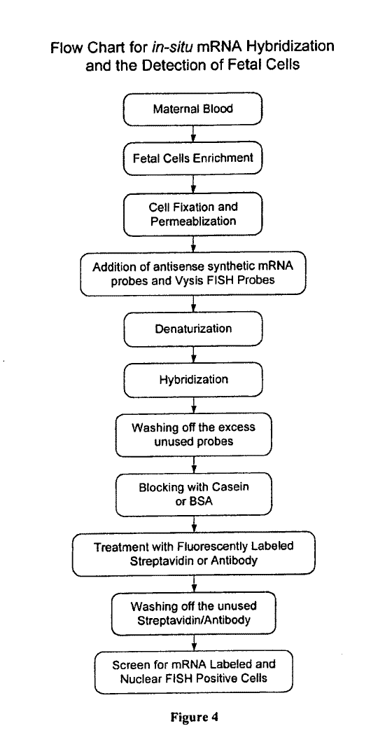

An exemplary flow chart for in situ mRNA hybridization and the subsequent detection of fetal cells is provided in FIG. 4. Blood samples were collected either in Cyto-Chex and/or TransFix (1:1). These cell stabilizers were indispensable for the stability of cells and their contents and were used according to manufacturer's instructions. The following steps were carried out in accordance with the flow chart.

Step 1: Fetal Cell Enrichment: Mononuclear cells from 10 ml of maternal blood collected in cell stabilizer(s) Cyto-Chex (Streck Labs) and/or TransFix (1;1) (Cytomark Ltd.) were isolated by density gradient centrifugation with Histopaque 1.083 to 1.190 (Sigma) or Percoll 1.13 according to the following protocol:

Added 1×PBS with 1% BSA to each blood tube to a final volume of 30 ml. Pipette up and down several times to mix.

Carefully layered the sample over 20 ml of Histopaque 1.083-1.119 or Percoll 1.13 using a 25 ml serological pipette making sure that Histopaque layer is not disturbed.

Centrifuged the sample tubes at 400×g for 45 minutes ensuring brake was off.

Removed and discarded the upper layer from each tube using a 25 ml serological pipette leaving behind approximately 1-2 ml of the top layer and without disrupting the monolayer at the interphase of the first and second layers.

Removed the interphase and Ficoll layers using a transfer pipette and carefully transferred into the appropriately labeled pre-blocked 50 ml tube. Left approximately 1 ml of Ficoll above the red blood cell (RBC) layer.
Aspirated from the top edge using a circular motion
Added PBS with 1% BSA to each tube (containing the interphase and Ficoll layers) using a 25 ml serological pipette to a final volume equal to 50 ml.

Pipetted up and down several times to mix.
Centrifuged the sample tubes at 400×g for 45 minutes with the brake set to 1 and the acceleration set to FAST.

Carefully aspirated the supernatant using the house vacuum aspiration system leaving approximately 5 ml in each tube.
Gently resuspended the pellet with a 1000 μl pipette (10 times, slowly).

Added PBS with 1% BSA using a 25 ml serological pipette to each tube to a final volume equal to 50 ml. Pipetted up and down several times to mix.
Centrifuged the sample tubes at 400×g for 45 minutes with the brake set to 1 and the acceleration set to FAST.

Carefully aspirated the supernatant using the house vacuum aspiration system leaving approximately 5 ml in each tube.

Carefully aspirated the remaining supernatant with a transfer pipette leaving approximately 800 μl above the cell pellet.

Step 2: Capture of Fetal Nucleated Red Blood Cells (fnRBCs): The cells from the above interphase and Ficoll layers were passed through a proprietary cell capture device that is coated With antibodies for fetal cell surface antigens. The cell capture devices are described in U.S. application Ser. Nos. 11/458,668 and 11/331,988, both of which are incorporated herein in their entirety. The antibodies used include, but are not limited to, anti-glycophorin A antibody (GPA), anti-transferrin-receptor antibody (CD71), anti-HLA-G233 antibody, and/or stem cells capture antibody such as anti-CD34 antibody. Typically, anti-glycophorin A antibody is linked to the cell capture device through a hydrophilic polymer. After cell capture, the majority red blood cells were lysed by exposing the device to 0.155 M NH4Cl for 1-2 minutes, followed by PBS washings to remove lysis solution.

Step 3: Cell Fixation and Permeabilization: The captured cells from step 2 above were treated with 4% paraformaldehyde in PBS at room temperature for 30 minutes.

After washing with PBS, the cells were treated with 0.5% Triton X-100 at RT for 10 minutes to permeabilize them. The cells were again washed with PBS and then dehydrated with sequential washings with 70%, 90% and 100% ethanol.

Step 4: In-Situ mRNA and Nuclear DNA (FISH) Hybridization: A hybridization buffer (HB) with the following composition was prepared:
50% Deionised formamide
0.3 M Sodium Chloride
20 mM Tris.HCl, pH 8.0
5 mM EDTA
10 mM Sodium Phosphate
10% Dextran
1×Denhardt's

mRNA and FISH Probes Cocktail:
30 uM stock of epsilon-hemoglobin probe=2 μl
30 uM stock of zeta-hemoglobin probe=2 μl
CEP-X Spectrum Green FISH probe=1 μl (Vysis)
CEP-Y Spectrum Orange FISH probe=1 μl (Vysis)
HB=14 μl
Total Volume=2 μl

In a typical in-situ hybridization experiment 20 μl of the above probes cocktail was added to the fixed cells inside the cell capture device.

Step 5: Denaturation: Denaturation was done by heating the device at 80° C. for 7 minutes.

Step 6: Hybridization: Hybridization was carried out at 37° C. to 45° C. for 4-14 hours.

Step 7: Washing: Post-hybridization washing of the excess unused probe was sequentially performed as below:
Wash i=50% Formamide in 2×SSC at 45° C. for 5 minutes
Wash ii=2×SSC at 45° C. for 2 minutes
Wash iii=2×SSC at RT for 5 minutes

Step 8: Blocking: To minimize the non-specific background, the cells were blocked either with 5% casein in PBS or with Perice's SuperBlock for 1 hour at 37° C.

Step 9: Visualization of Fetal mRNA and Nuclear Chromosomal DNA: After washing off the blocking solution from step 8 with 2×SSC, the cells were either treated with a solution of 1 μg/μl streptavidin (for biotinylated probes) or anti-DIG (for DIG labeled probes) that is conjugated to a fluorophore of choice, namely, Texas Red, Alexa fluor, etc. This treatment was done for one hour at 37° C. after which the excess reagents was washed off by multiple (3-5) washings with 2×SSC at 37° C.

Stabilization of DIG—Anti-DIG Complex: It was necessary to stabilize the DIG-Anti-DIG complex by cross-linking it with cross-linkers such as 4% paraformaldehyde, or other bifunctional cross-linkers, such as Pierce's Sulfo-EGS (ethylene glycol bis(succinimidylsuccinate)). Other cross-linkers useful in retaining mRNA signal can be of the general structure: R—(CH2-CH2-O)ₙ—R, where n=2-80 and R=Sulfo-NHS, imidoester.

Without this step, the mRNA signal weakened with time. We attributed this to the instability of anti-DIG—DIG complex in the presence of DAPI-antifade solution which we apply to the cells to visualize their nuclei after FISH.

Typically, 1-5% solution of the cross-linker in PBS at pH 7.0 was reacted with the cells at RT for 1 hour. The excess linker was then washed off.

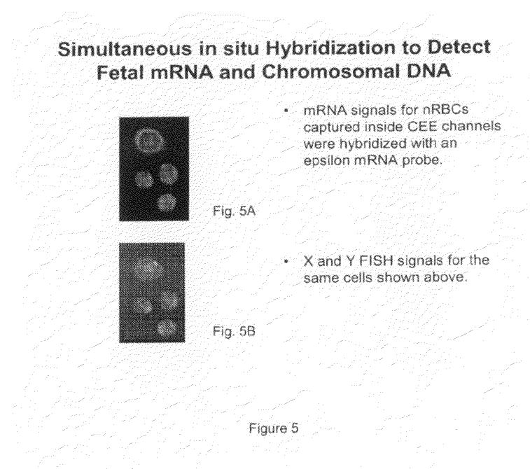

Step 10: Visualization of mRNA and FISH Signal: After introducing 20 μl of commercial DAPI-antifade solution, the cells were analyzed under a fluorescence Microscope. Typical fluorescence pictures of fetal cells that are both mRNA and FISH positive are shown in FIGS. 5A and 5B.

Although the invention has been described with reference to the presently preferred embodiment, it should be understood that various changes and modifications, as would be obvious to one skilled in the art, can be made without departing from the spirit of the invention. Accordingly, the invention is limited only by the following claims.

1. A method for detecting mRNA and chromosomal DNA in a cell comprising:
hybridizing in situ a first probe and a second probe to a biological sample containing one or more target cells in a condition allowing the first probe to specifically hybridize to its mRNA target in a target cell and the second probe to specifically hybridize to its chromosomal DNA target in the target cell,
wherein the first probe has a Tm of at least about 80° C. or a GC content of at least about 40%.

2. The method of claim 1, wherein the first probe is a probe for an mRNA marker of the target cell.

3. The method of claim 1, wherein the target cell is a fetal cell.

4. The method of claim 1, wherein the first probe is a probe for an mRNA marker of a fetal cell.

5. The method of claim 1, wherein the first probe is a probe for an mRNA marker of a fetal cell selected from the group consisting of epsilon-hemoglobin mRNA; zeta-hemoglobin mRNA; gamma-hemoglobin mRNA; Y-chromosome-specific zinc finger protein mRNA (ZFY-mRNA); fetal alpha-fetoprotein (AFP) mRNA; gamma-glutamyl-transpeptidase (GGT) mRNA; human placental lactogen 1, 2, 3, and 4 (hPL, aka PLAC-1-4) mRNA; pregnancy associated plasma protein-A (PAPP-A) mRNA; corticotropin hormone-releasing factor (CRH) mRNA; tissue factor pathway inhibitor 2 (TFPI-2) mRNA; KISS-1 metastatic-supressor mRNA; and PLAC-1 mRNA; pregnancy specific beta-1-glycoprotein-2, 3, 4, 5, 6, 7 and 9 mRNAs; chorionic somatomammotropin hormone 2 and variants (v1, v2, v3, v4, v5) mRNAs; growth hormone variant mRNA; alfa-disintegrin and metalloproteinase domain variants mRNAs; dipeptidylpeptidase 7 mRNA; fibulin mRNA; s100 calcium binding protein mRNA; prostate differentiation factor mRNA; and cytochrome P450, subfamily xix mRNA.

6. The method of claim 1, wherein the first probe comprises one or more modified nucleotides.

7. The method of claim 1, wherein the first probe comprises at least about 30% modified nucleotides.

8. The method of claim 1, wherein the first probe comprises one or more modified nucleotides selected from the group consisting of locked nucleic acid (LNA), peptide nucleic acid (PNA), nucleic acid containing 2′-OMe nucleotides, nucleic acid containing methylphosphonates, nucleic acid containing phosphorothioates, nucleic acid containing morpholino and combinations thereof.

9. The method of claim 1, wherein the first probe comprises about 20 to about 50 nucleotides.

10. The method of claim 1, wherein the first probe is labeled with a detectable entity.

11. The method of claim 1, wherein the first probe is labeled with biotin.

12. The method of claim 1, wherein the second probe is a fluorescently labeled probe for in-situ hybridization.

13. The method of claim 1, wherein the biological sample is a blood or a cervical mucous sample.

14. The method of claim 1, wherein the target cell is a fetal cell from a fetus and the biological sample is a blood sample from a maternal host of the fetus.

15. The method of claim 1, wherein the target cell is a fetal cell from a fetus and the biological sample is a fetal cell enrichment sample obtained from the blood sample of a maternal host of the fetus.

16. The method of claim 1, wherein the target cell is a neoplastic cell.

17. The method of claim 1, wherein cells within the biological sample are treated to be suitable for in situ hybridization.

18. The method of claim 1, wherein cells within the biological sample are treated with about 4% paraformaldehyde in PBS at room temperature for at least 30 minutes.

19. The method of claim 1, wherein the first probe and the second probe are incubated with the biological sample in a denaturing condition prior to the hybridization.

20. The method of claim 1, wherein the condition comprises hybridizing at about 37° C. to about 45° C. for about 4 hours to about 14 hours.

21. The method of claim 1, wherein a wash process is carried out after the hybridization of the first probe and the second probe and wherein the wash process contains a wash at a temperature varying from room temperature to 80° C.

22. The method of claim 1 wherein the wash solution comprises 1×SSC, formamide and optionally, one or more detergents.

23. The method of claim 1, wherein one or more blocking agents are added prior to detecting the hybridization of the first probe to its mRNA target and the second probe to its chromosomal DNA target.

24. The method of claim 1, wherein the target cell is a fetal cell and the first probe comprises a nucleic acid sequence selected from the group consisting of SEQ ID NO:1-SEQ ID NO: 40.

25. The method of claim 1, wherein the hybridization of the first probe to its mRNA target and the second probe to its chromosomal DNA target are carried out concurrently.

26. The method of claim 1, wherein the hybridization of the first probe to its mRNA target and the second probe to its chromosomal DNA target are detected concurrently.

27. A method for detecting a genetic disorder of a fetus comprising
hybridizing in situ a first probe and a second probe to a biological sample containing one or more fetal cells from the fetus in a condition allowing the first probe to specifically hybridize to its mRNA target in a fetal cell and the second probe to specifically hybridize to its chromosomal DNA target in the fetal cell,
wherein the first probe has a Tm of at least about 80° C. or a GC content of at least about 40%, and
wherein hybridization of the first probe to its mRNA target and the second probe to its chromosomal DNA target is indicative of a genetic disorder of the fetus.

28. The method of claim 27, wherein the genetic disorder is selected from the group consisting of Cystic Fibrosis, Sickle-Cell Anemia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, and RhD Syndrome.

29. The method of claim 27, wherein the biological sample is a blood sample from a maternal host of the fetus.

30. The method of claim 27, wherein the biological sample is a fetal cell enrichment sample obtained from a biological sample of the maternal host of the fetus.

31. The method of claim 27, wherein the chromosomal DNA target is associated with a genetic disorder selected from the group consisting of Cystic Fibrosis, Sickle-Cell Anemia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, and RhD Syndrome.

32. The method of claim 27, wherein the mRNA target is an mRNA marker of a fetal cell.

33. The method of claim 27, wherein the fetus is at least seven weeks old.

34. A probe comprising a nucleic acid sequence selected from the group consisting of SEQ ID NOs 1-40.

35. The probe of claim 34 wherein it contains one or more modified nucleotides.

36. The probe of claim 34 wherein the it comprises at least about 30% modified nucleotides.

37. The probe of claim 34 wherein it comprises one or more modified nucleotides selected from the group consisting of locked nucleic acid (LNA), peptide nucleic acid (PNA), nucleic acid containing 2′-OMe nucleotides, nucleic acid containing methylphosphonates nucleic acid containing phosphorothioates, nucleic acid containing morpholino analogs and combinations thereof.

38. The probe of claim 34 wherein it comprises about 20 to about 50 nucleotides.

39. The probe of claim 34 wherein it is labeled with a detectable entity.

40. The probe of claim 34 wherein it is labeled with a direct or indirect detectable entity which emits light through an excitation-emission mechanism or through a chemiluminescence mechanism.

41. The probe of claim 34 wherein it is labeled with a direct label selected from the group consisting of fluorescein, Cy3, Cy5, Cy7, Texas Red, StarBright Green, StarBright Orange, rhodamine green, Oregon Green, bodipy fluorophores, Alexa Fluor dyes with emission between 350 and 750 nm, and combinations thereof.

42. The probe of claim 34 wherein it is labeled with an indirect label selected from the group consisting of digoxigenin, biotin, Lucifer yellow, dinitrophenyl (DNP), dansyl, and combinations thereof.

43. A kit comprising the probe of claim 34.

44. The kit of claim 43 further comprising a second probe for a chromosomal DNA target in a fetal cell.

45. The kit of claim 43 further comprising a second probe for a chromosomal DNA target associated with a genetic disorder in a fetus.

46. The kit of claim 43 further comprising a buffer suitable for hybridization of the probe to its mRNA target.

47. The kit of claim 43 further comprising a second probe for a chromosomal DNA target in a fetal cell and a buffer suitable for hybridization of the probe to its mRNA target and the second probe to its chromosomal DNA target.

48. The kit of claim 47 further comprising a wash buffer and a blocking agent.

49. The kit of claim 48 further comprising a reagent for treating a cell sample so that the sample is suitable for in situ hybridization.


SEQUENCE LISTING (40 sequences)

SEQ ID NO: 1 - DNA, 26 bp, Homo sapiens
  zeta-globin probe
attggcgcattacgcggtaagtgggg

SEQ ID NO: 2 - DNA, 26 bp, Homo sapiens
  zeta-globin probe
ttggtcagagacatggcggcagggtg

SEQ ID NO: 3 - DNA, 26 bp, Homo sapiens
  zeta-globin probe
gataggaacttgtcccaggcggcgtg

SEQ ID NO: 4 - DNA, 26 bp, Homo sapiens
  zeta-globin probe
ctctccagagtctcggtgccgatggt

SEQ ID NO: 5 - DNA, 26 bp, Homo sapiens
  zeta-globin probe
gccgccgatgtcgtcgatgctcttca

SEQ ID NO: 6 - DNA, 26 bp, Homo sapiens
  zeta-globin probe
acgaccgataggaacttgtcccaggc

SEQ ID NO: 7 - DNA, 26 bp, Homo sapiens
  zeta-globin probe
atgtcgtcgatgctcttcaccgcgtc

SEQ ID NO: 8 - DNA, 26 bp, Homo sapiens
  zeta-globin probe
cgctcagcggtacttctcggtcagga

SEQ ID NO: 9 - DNA, 26 bp, Homo sapiens
  zeta-globin probe
gccgccgatgtcgtcgatgctcttca

SEQ ID NO: 10 - DNA, 26 bp, Homo sapiens
  zeta-globin probe
taagtggggctggggaacctccagga

SEQ ID NO: 11 - DNA, 26 bp, Homo sapiens
  epsilon-globin probe
atgtgcagaaggagggtgtcagggtc

SEQ ID NO: 12 - DNA, 26 bp, Homo sapiens
  epsilon-globin probe
caccatgatgccaggcctgagagctt

SEQ ID NO: 13 - DNA, 26 bp, Homo sapiens
  epsilon-globin probe
caggtccggagagggtcagcagtgat

SEQ ID NO: 14 - DNA, 26 bp, Homo sapiens
  epsilon-globin probe
gcttagcaaaggcgggcttgaggttg

SEQ ID NO: 15 - DNA, 26 bp, Homo sapiens
  epsilon-globin probe
catgtgcagaaggagggtgtcagggt

SEQ ID NO: 16 - DNA, 26 bp, Homo sapiens
  epsilon-globin probe
tcacaggaacacctgcaaactggaag

SEQ ID NO: 17 - DNA, 26 bp, Homo sapiens
  epsilon-globin probe
gataatgtgtgctggatcctgaggct

SEQ ID NO: 18 - DNA, 26 bp, Homo sapiens
  epsilon-globin probe
agtagccagaataatcaccatcacgt

SEQ ID NO: 19 - DNA, 26 bp, Homo sapiens
  epsilon-globin probe
agtaaaatgcaccatgatgccaggcc

SEQ ID NO: 20 - DNA, 26 bp, Homo sapiens
  epsilon-globin probe
caggtccggagagggtcagcagtgat

SEQ ID NO: 21 - DNA, 26 bp, Homo sapiens
  gamma-globulin 1 probe
tgaatcatgggcagtgagctcagtgg

SEQ ID NO: 22 - DNA, 26 bp, Homo sapiens
  gamma-globulin 1 probe
atgggcagtgagctcagtggtatctg

SEQ ID NO: 23 - DNA, 26 bp, Homo sapiens
  gamma-globulin 1 probe
cactggccactgcagtcaccatcttc

SEQ ID NO: 24 - DNA, 26 bp, Homo sapiens
  gamma-globulin 1 probe
aggaagcctgcacctcaggggtgaat

SEQ ID NO: 25 - DNA, 26 bp, Homo sapiens
  gamma-globulin 1 probe
tagacaaccaggagccttcccagggt

SEQ ID NO: 26 - DNA, 26 bp, Homo sapiens
  gamma-globulin 1 probe
tgaatcatgggcagtgagctcagtgg

SEQ ID NO: 27 - DNA, 26 bp, Homo sapiens
  gamma-globulin 1 probe
cagtggtatctggaggacagggcact

SEQ ID NO: 28 - DNA, 26 bp, Homo sapiens
  gamma-globulin 1 probe
caggggtgaattctttgccgaaatgg

SEQ ID NO: 29 - DNA, 26 bp, Homo sapiens
  gamma-globulin 1 probe
gccttcccagggtttctcctccagca

SEQ ID NO: 30 - DNA, 26 bp, Homo sapiens
  gamma-globulin 1 probe
caccagcacatttcccaggagcttga

SEQ ID NO: 31 - DNA, 26 bp, Homo sapiens
  gamma-globulin 2 probe
tagacaaccaggagccttcccagggt

SEQ ID NO: 32 - DNA, 26 bp, Homo sapiens
  gamma-globulin 2 probe
acagggcactggccactccagtcacc

SEQ ID NO: 33 - DNA, 26 bp, Homo sapiens
  gamma-globulin 2 probe
ctctgcatcatgggcagtgagctcag

SEQ ID NO: 34 - DNA, 26 bp, Homo sapiens
  gamma-globulin 2 probe
cgaaatggattgccaaaacggtcacc

SEQ ID NO: 35 - DNA, 26 bp, Homo sapiens
  gamma-globulin 2 probe
ccacattcaccttgccccacaggctt

SEQ ID NO: 36 - DNA, 26 bp, Homo sapiens
  gamma-globulin 2 probe
gccttcccagggtttctcctccagca

SEQ ID NO: 37 - DNA, 26 bp, Homo sapiens
  gamma-globulin 2 probe
agggcactggccactccagtcaccat

SEQ ID NO: 38 - DNA, 26 bp, Homo sapiens
  gamma-globulin 2 probe
gtagacaaccaggagccttcccaggg

SEQ ID NO: 39 - DNA, 26 bp, Homo sapiens
  gamma-globulin 2 probe
caccagcacatttcccaggagcttga

SEQ ID NO: 40 - DNA, 26 bp, Homo sapiens
  gamma-globulin 2 probe
caggggtgaattctttgccgaaatgg